(19) United States
(12) Patent Application Publication
(10) Pub. No.: US 2023/0139402 A1
(43) Pub. Date: May 4, 2023

(54) SYSTEMS AND METHODS FOR REGISTRATION FEATURE INTEGRITY CHECKING

(71) Applicant: INTUITIVE SURGICAL OPERATIONS, INC., Sunnyvale, CA (US)

(72) Inventors: Dinesh RABINDRAN, San Jose, CA (US); Simon P. DIMAIO, San Carlos, CA (US); Changyeob SHIN, Los Angeles, CA (US); Kollin M. TIERLING, Los Altos Hills, CA (US)

(21) Appl. No.: 17/800,524

(22) PCT Filed: Feb. 23, 2021
(86) PCT No.: PCT/US2021/019219

§ 371 Date: Aug. 17, 2022

Related U.S. Application Data
(60) Provisional application No. 62/980,956, filed on Feb. 24, 2020.

(51) Int. Cl. G06V 10/98; G06T 7/246; G06T 1/00; G06T 7/33; G06V 10/77; A61B 1/00; B25J 9/16

(52) U.S. Cl. CPC G06V 10/98; G06T 7/246; G06T 1/0014; G06T 7/337; G06V 10/7715; A61B 1/00149; B25J 9/1697; G06T 2207/10068

(57) ABSTRACT

Systems and methods for registration feature integrity checking include a repositionable arm configured to support a repositionable device and a control unit. The control unit is configured to receive a feature set including one or more features extracted from one or more images of a repositionable structure obtained from an imaging device, determine an expected feature corresponding to an extracted feature in the feature set based on one or more models of the repositionable structure, determine an error between the extracted feature and the expected feature, determine whether to remove the extracted feature from the feature set based on the determined error, remove the extracted feature from the feature set in response to determining that the extracted feature should be removed from the feature set, and provide the feature set to a registration module. The repositionable structure includes the repositionable arm and/or the repositionable device.

### RELATED APPLICATIONS

This application claims the benefit of U.S. Provisional Application No. 62/980,956, filed Feb. 24, 2020, which is incorporated by reference herein.

### TECHNICAL FIELD

The present disclosure relates generally to operation of devices having instruments mounted to repositionable arms of computer-assisted devices and more particularly to determining perform integrity checking on features used to support registration between instruments on different computer-assisted devices.

### BACKGROUND

More and more devices are being replaced with computer-assisted electronic devices. This is especially true in industrial, entertainment, educational, and other settings. As a medical example, many hospitals of modern day contain large arrays of electronic devices in operating rooms, interventional suites, intensive care wards, emergency rooms, laboratories, examination rooms, and/or the like. For example, glass and mercury thermometers are being replaced with electronic thermometers, intravenous drip lines now include electronic monitors and flow regulators, and traditional hand-held surgical and other medical instruments are being replaced by computer-assisted medical devices.

These computer-assisted devices are useful for performing operations and/or procedures on materials, such as the tissue of a patient, that are located in a workspace. When the workspace is separated from the operator controlling the computer-assisted device, it is common for the operator to control the computer-assisted device using teleoperation and to monitor the activity of the computer-assisted device using an imaging device positioned to capture images or video of the workspace. In computer-assisted devices with instruments that are mounted to repositionable arms and/or manipulators, the teleoperation typically involves the operator using one or more input controls to provide movement commands for the instruments that are, for example, implemented by driving one or more joints in a respective repositionable arm and/or manipulator. In some computer-assisted devices, one of the instruments may be an imaging device mounted to a repositionable arm so that the operator may change a location and/or a direction of a field of view of the imaging device so as to be able to capture images of the workspace from different positions and orientations.

Because the operator is relying on images of the other instruments captured by the imaging device to teleoperate the other instruments, it is useful to know the geometric relationships (e.g., the registrations) between the imaging device and the other instruments so that movements of the hands of the operator relative to an eye coordinate system of an operator station may be properly translated to motions of the other instruments. However, like many techniques, the quality and/or accuracy of the registration depends significantly on the quality and/or accuracy of the data used to determine the registration transform.

Accordingly, it would be advantageous to have methods and systems to determine whether information and/or features being used to establish registration between an imaging device and an instrument have sufficient integrity to provide an appropriately accurate registration.

### SUMMARY

Consistent with some embodiments, a computer-assisted system includes a repositionable arm configured to support a repositionable device and a control unit coupled to the repositionable arm. The control unit is configured to receive, from an image processing system or a feature extraction system, a feature set comprising one or more extracted features, the one or more extracted features being extracted from one or more images of a repositionable structure obtained from an imaging device, wherein the repositionable structure comprises at least one component selected from the group consisting of: the repositionable arm and the repositionable device; determine, based on one or more models of the repositionable structure, a first expected feature, the first expected feature corresponding to a first extracted feature in the feature set; determine an error between the first extracted feature and the first expected feature; determine whether to remove the first extracted feature from the feature set based on the determined error; in response to determining that the first extracted feature should be removed from the feature set, remove the first extracted feature from the feature set; provide the feature set to a registration module; and cause motion of the repositionable device by commanding movement of the repositionable arm based at least on a registration provided by the registration module.

Consistent with some embodiments, a method performed by a control unit includes receiving, from an image processing system or a feature extraction system, a feature set comprising one or more extracted features, the one or more extracted features being extracted from one or more images of a repositionable structure, the one or more images being obtained from an imaging device, wherein the repositionable structure comprises at least one component selected from the group consisting of: a repositionable arm and a repositionable device supported by the repositionable arm; determining, based on one or more models of the repositionable structure, a first expected feature corresponding to a first extracted feature in the feature set; determining an error between the first extracted feature and the first expected feature; determining whether to remove the first extracted feature from the feature set based on the determined error; in response to determining that the first extracted feature should be removed from the feature set, removing the first extracted feature from the feature set; and providing the feature set to a registration module.

Consistent with some embodiments, a non-transitory machine-readable medium including a plurality of machine-readable instructions which when executed by one or more processors are adapted to cause the one or more processors to perform any of the methods described herein.

### BRIEF DESCRIPTION OF THE DRAWINGS

FIG. 1 is a simplified diagram of a computer-assisted system according to some embodiments.

FIG. 2 is a simplified diagram of a control system according to some embodiments.

FIG. 3 is a simplified diagram of a data flow model for an image-based registration system according to some embodiments.

FIG. 4 is a simplified diagram of a distal end of an instrument illustrating exemplary features according to some embodiments.

FIG. 5 is a simplified diagram of a method of feature integrity checking according to some embodiments.

In the figures, elements having the same designations have the same or similar functions.

### DETAILED DESCRIPTION

This description and the accompanying drawings that illustrate inventive aspects, embodiments, implementations, or modules should not be taken as limiting—the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, or module may, whenever practical, be included in other embodiments, implementations, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw, angle-axis, rotation matrix, quaternion representation, and/or the like). As used herein, the term “shape” refers to a set positions or orientations measured along an element. As used herein, and for a device with repositionable arms, the term “proximal” refers to a direction toward the base of the computer-assisted device along its kinematic chain and “distal” refers to a direction away from the base along the kinematic chain. As used herein, the term “pose” refers to the six degree of freedom (DOF) spatial position and orientation of a coordinate system of interest attached to a rigid body.

As used herein, the term “feature” refers to a point, a distance, an axis or direction, an angle between other features, and/or similar geometrical elements, either measured directly using an imaging device or derived from such measurements using mathematical models.

As used herein, the term “kinematic-invariant feature” refers to a feature that does not change even though the positions and/or orientations of one or more joints of the device to which the feature pertains change. The term “kinematic-variant feature” refers to a feature that may be changed as a result of a change in the positions and/or orientations of the one or more joints of the device to which the feature pertains. The terms “position-invariant,” “rotation-invariant,” and “pose-invariant” refer to a feature whose position, orientation, or pose, respectively, do not change over time even though the device to which the feature pertains may change its position, orientation, or pose over time.

As used herein, the terms “observed feature” or “primary feature” refer to a feature that is measured or detected directly by an imaging device, such as a one-dimensional, two-dimensional (2D), three-dimensional (D), or other-dimensional feature that may be observed in a left and/or right image captured by a stereoscopic imaging device. The terms “derived feature,” “secondary feature,” or “computed feature” refer to a feature that is estimated or computed from one or more other primary and/or secondary features using mathematical models and/or multi-view calibration parameters of the imaging device, and may include one-dimensional, two-dimensional (2D), three-dimensional (D), or other-dimensional features. A collection of one or more features, primary and/or secondary, is termed a feature set. The term “confidence level” refers to a confidence with which a primary feature is determined or detected, and the term “confidence score” refers to a confidence with which a secondary feature is estimated or computed.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an implementation using a surgical system, such as the da Vinci® Surgical System commercialized by Intuitive Surgical, Inc. of Sunnyvale, Calif. Knowledgeable persons will understand, however, that inventive aspects disclosed herein may be embodied and implemented in various ways, including robotic and, if applicable, non-robotic embodiments and implementations. Implementations on da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (without return to a human or animal anatomy) and for procedures on human or animal cadavers. Further, these techniques can also be used for medical treatment or diagnosis procedures that include, or do not include, surgical aspects.

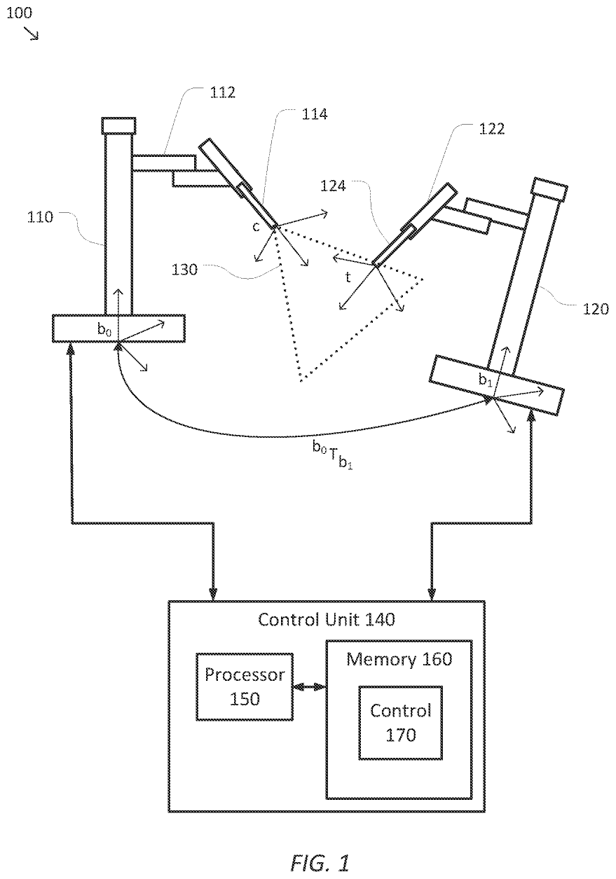

FIG. 1 is a simplified diagram of a computer-assisted system 100 according to some embodiments. As shown in FIG. 1, computer-assisted system 100 includes two computer-assisted devices 110 and 120. Computer-assisted device 110 includes a repositionable structure having a repositionable arm 112 configured to support an instrument. The instrument may be an imaging instrument, a manipulation instrument, an irrigation or suction instrument, or any other appropriate instrument. In the example of FIG. 1, instrument comprises an imaging device 114. In some examples, the instrument comprises an imaging device 114 that is a monoscopic or stereoscopic camera, a still or video camera, an endoscope, a hyperspectral device, an infrared or ultrasonic device, an ultrasonic device, a fluoroscopic device, and/or the like. Similarly, computer-assisted device 120 includes a repositionable structure having a repositionable arm 122 configured to support an instrument 124. In some examples, instrument 124 may be an imaging device, a non-imaging device, and/or the like. In some medical examples, the instrument may be a medical instrument, such as forceps, clip appliers, a gripper, a retractor, a cautery instrument, a suction instrument, a suturing device, a stapling device, a cutting device, and/or the like. In some examples, instrument 124 may include an end effector capable of performing multiple tasks, such as both grasping a material (e.g., tissue of a patient) located in the workspace and delivering energy to the grasped material. In some examples, the energy may include ultrasonic, radio frequency, electrical, magnetic, thermal, light, and/or other types of energy. In some examples, each of repositionable arm 112, repositionable arm 122, imaging device 114, and/or instrument 124 may include one or more joints. In some medical examples, imaging device 114 and/or instrument 124 may be inserted into a workspace (e.g., anatomy of a patient or cadaver, a veterinary subject, an anatomical model, and/or the like) through a respective cannula, access port, and/or the like. In some medical examples, imaging device 114 may captures images outside of a workspace of the instrument 124, such as an exterior anatomy (e.g., exterior anatomy of a patient, cadaver, veterinary subject, and/or the like), part or all of one or more computer-assisted devices, repositionable arms, repositionable devices, personnel or equipment near the imaging device 114, and/or the like. In some examples, imaging device 114 may capture images of anatomy and/or structures both interior and exterior to the anatomy. In some embodiments, computer-assisted system 100 may be found in an operating room and/or an interventional suite.

FIG. 1 also shows a field of view 130 of imaging device 114 by indicating a region within the workspace where imaging device 114 may capture images of the workspace and objects and events within the workspace. In the configuration shown in FIG. 1, at least a distal portion of instrument 124 is located within field of view 130, allowing imaging device 114 to capture images of at least the distal portion of instrument 124 when instrument 124 is not visually obstructed completely from imaging device 114. According to some embodiments and as described further below, images of instrument 124 obtained by imaging device 114 may be used to determine a registration transform between computer-assisted device 110 and computer-assisted device 120.

FIG. 1 also shows various coordinate systems that are useful in describing how the registration transform is determined. Coordinate system b₀ corresponds to a base coordinate system of computer-assisted device 110. In some examples, coordinate system b₀ may have its origin located at a central point on a base of computer-assisted device 110 and may be aligned with one or more major axes of computer-assisted device 110. In some examples, the central point on the base of computer-assisted device 110 may be on a level, planar surface on which computer-assisted device 110 may be wheeled, slid, and/or otherwise repositioned. In some examples, a z-axis of coordinate system b₀ may correspond to a vertical up direction. Similarly, coordinate system b₁ corresponds to a base coordinate system of computer-assisted device 120. In some examples, coordinate system b₁ may have its origin located at a central point on a base of computer-assisted device 120 and may be aligned with one or more major axes of computer-assisted device 120. In some examples, the central point on the base of computer-assisted device 120 may be on a level, planar surface on which computer-assisted device 120 may be wheeled, slid, and/or otherwise repositioned. In some examples, a z-axis of coordinate system b₁ may correspond to a vertical up direction. In some embodiments, coordinate systems b₀ and/or b₁ need not be necessarily be on the same or parallel planes, such as in the case when one or more of the computer-assisted devices 110 and/or 120 are mounted on a table, a wall, a ceiling, and/or the like.

FIG. 1 also shows an imaging device coordinate system c (e.g., for camera) that may be used to describe positions and/or orientations of objects (e.g., the distal end of instrument 124) in images captured by imaging device 114. In some examples, an origin of coordinate system c may be located at a distal end of imaging device 114. In some examples, the origin of coordinate system c may be located at a mid-point of an imaging plane associated with imaging device 114. In some examples, a z-axis of coordinate system c may be oriented in a direction of view of imaging device 114. In some examples, imaging device 114 may be a steerable imaging device and/or a flexible continuum-robot like imaging device, such that coordinate system c may be at or near a distal tip of the imaging device. In some examples, the coordinate system c may not be attached to a physical point and may be virtually coupled, rigidly or otherwise, to a reference point on the imaging device.

FIG. 1 also shows an instrument coordinate system t (e.g., for tool) that may be used to describe positions and/or orientations of a constellation of points on or near instrument 124 relative to a known fixed point on instrument 124. In some examples, one or more kinematic models of computer-assisted device 120 and/or instrument 124 may be used to determine a pose of instrument coordinate system t relative to the base coordinate system b₁ of computer-assisted device 120. In some examples, the fixed point may correspond to a distal end of instrument 124. In some examples, the fixed point may correspond to an easily identified point feature on instrument 124, such as a fiducial, an indicia mark (including markings for indicating instrument type or source), a representative structure such as a tool tip or a clevis pin about which one or more jaws and/or links of instrument 124 may be rotated, a point at a predefined displacement from any of the above described fixed points, and/or the like. In some examples, a principal axis of coordinate system t may correspond to an alignment feature of instrument 124. An alignment feature could be a real or virtual line of symmetry, a line associated with a principal rotational and/or translational motion of instrument 124, a line integral to the shape of instrument 124, and/or the like. In some examples, the alignment feature may be an axis determined from a fiducial indicating an axis, an indicia mark indicating an axis, an axis of a shaft of instrument 124, an axis corresponding to a distinctive structure (e.g., as located between two jaws of instrument 124 and oriented with the grasping faces of the two jaws while the jaws are closed), an axis formed by a line segment connecting any of the fixed points described above, a virtual axis at a predefined origination relative to any of the above described alignment features or axes, and/or the like. In some examples, coordinate system t may not be attached to a physical point and may be virtually coupled, rigidly or otherwise, to a reference point and/or the fixed point on instrument 124.

As is further shown in the embodiments of FIG. 1, because the bases of both computer-assisted device 110 and computer-assisted device 120 may be moved and/or oriented relative to and independently of each other (e.g., they have independent kinematic chains), the geometric relationship between coordinate system b₀ and coordinate system b₁ is subject to change. In some examples, the geometric relationship between coordinate system b₀ and coordinate system b₁ may be characterized using a 6 degrees of freedom (6 DOF) registration transform ^{b}^{₀}T_{b}_{₁}. As shown in Equation 1, registration transform ^{b}^{₀}T_{b}_{₁} includes a 3 degrees of freedom (3 DOF) rotation portion ^{b}^{₀}R_{b}_{₁} and a 3 DOF translation portion ^{b}^{₀}T_{b}_{₁} Rotation portion ^{b}^{₀}R_{b}_{₁} is a 3×3 matrix describing the 3-dimensional rotational difference between coordinate system b₀ and coordinate system b₁. In some examples, rotation portion ^{b}^{₀}R_{b}_{₁} describes rotation about the three axes of the coordinate system. In some examples, the three axes may correspond to x, y, and z axes; roll, pitch, and yaw axes, and/or the like. In some examples, the rotation portion may be represented using angle-axis, quaternion, and/or similar equivalent notations. Translation portion ^{b}^{₀}T_{b}_{₁} is a 3×1 vector describing the 3-dimensional displacement between coordinate system b₀ and coordinate system b₁. In some examples, registration transform ^{b}^{₀}T_{b}_{₁} may be used to determine the full transform from instrument coordinate system t to imaging device coordinate system c (e.g., ^{c}Tₜ). In some examples, registration transform ^{b}^{₀}T_{b}_{₁} may be defined as a correction to the assumed imaging device coordinate system c (e.g., ^{c′}T_{c}) where c′ is the corrected coordinated system of imaging device 114 that aligns the motion of instrument 124 and motion of the one or more input controls used to control instrument 124.

$${}_{}^{{b}_{0}} {T}_{{b}_{1}}^{} = [ \begin{matrix}{}_{}^{{b}_{0}} {R}_{{b}_{1}}^{} & {}_{}^{{b}_{0}} {t}_{{b}_{1}}^{} \\ 0 & 1\end{matrix} ] \qquad Equation 1$$

In some embodiments, when computer-assisted device 110 and computer-assisted device 120 are located on a common planar surface and have a common vertical up direction (e.g., both coordinate system b₀ and coordinate system b₁ are located on a same level reference plane and have a same vertical up axis), reference transform ^{b}^{₀}R_{b}_{₁} may be simplified to a single rotation about the vertical up axis and ^{b}^{₀}T_{b}_{₁} may be simplified to a 2D lateral translation between coordinate system b₀ and coordinate system b₁. In some examples, when the bases of computer-assisted devices 110 and 120 are on parallel planes, each orthogonal to the vertical up axis and separated by a distance, reference transform ^{b}^{₀}R_{b}_{₁} may be simplified to a single rotation about the vertical up axis and ^{b}^{₀}T_{b}_{₁} may be a 3D translation vector.

In some embodiments, where the 6 DOF registration transform needs to be determined, the inclination or elevation angle between the bases of computer-assisted devices 110 and 120 may be known through one or more inclinometers and/or accelerometers, thus reducing the number of DOFs of registration transform ^{b}^{₀}T_{b}_{₁} that need to be computed by other means.

Computer-assisted device 110 and computer-assisted device 120 are both coupled to a control unit 140 via respective interfaces. Each of the respective interfaces may include one or more cables, connectors, and/or buses and may further include one or more networks with one or more network switching and/or routing devices. Control unit 140 includes a processor 150 coupled to memory 160. Operation of control unit 140 is controlled by processor 150. And although control unit 140 is shown with only one processor 150, it is understood that processor 150 may be representative of one or more central processing units, multi-core processors, microprocessors, microcontrollers, digital signal processors, field programmable gate arrays (FPGAs), application specific integrated circuits (ASICs), graphics processing units (GPUs), tensor processing units (TPUs), and/or the like in control unit 140. Control unit 140 may be implemented as a stand-alone subsystem and/or as a board added to a computing device or as a virtual machine.

Memory 160 may be used to store software executed by control unit 140 and/or one or more data structures used during operation of control unit 140. Memory 160 may include one or more types of machine-readable media. Some common forms of machine readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

As shown, memory 160 includes a control module 170 that is responsible for controlling one or more aspects of the operation of computer-assisted device 110 and/or computer-assisted device 120 including, for example, the control of movement and/or operation of each of computer-assisted device 110, computer-assisted device 120, repositionable arm 112, repositionable arm 122, imaging device 114, and/or instrument 124; determining a registration transform between computer-assisted device 110 and computer-assisted device 120, and/or the like as is described in further detail below. And although control module 170 is characterized as a software module, control module 170 may be implemented using software, hardware, and/or a combination of hardware and software.

As discussed above and further emphasized here, FIG. 1 is merely an example which should not unduly limit the scope of the claims. One of ordinary skill in the art would recognize many variations, alternatives, and modifications. According to some embodiments, computer-assisted system 100 may further include one or more operator stations having respective one or more input controls for determining a desired motion for computer-assisted device 110, computer-assisted device 120, repositionable arm 112, repositionable arm 122, imaging device 114, and/or instrument 124. In some examples, the operator station is in the form of a console. In some examples, a single operator station may be shared between computer-assisted device 110 and computer-assisted device 120. In some examples, computer-assisted device 110 and computer-assisted device 120 may have separate operator stations. In some examples, the one or more operator stations may be coupled to computer-assisted device 100, computer-assisted device 120, and/or control unit 140 using respective interfaces. Each of the respective interfaces may include one or more cables, connectors, and/or buses and may further include one or more networks with one or more network switching and/or routing devices.

According to some embodiments, computer-assisted system 100 may include any number of computer-assisted devices with corresponding repositionable arms and/or instruments of similar or different in design from computer-assisted device 110 and/or computer-assisted device 120. In some examples, each of the computer-assisted devices may include two, three, four, or more repositionable arms and/or instruments.

According to some embodiments, each of computer-assisted devices 110 and/or 120 may have separate control units similar to control unit 140. In some examples, each of the separate control units may be coupled to each other via an interface. The interface may include one or more cables, connectors, and/or buses and may further include one or more networks with one or more network switching and/or routing devices. In some examples, the separate control units may use the interface to share and/or exchange information about their respective computer-assisted devices. In some examples, the exchanged information may include kinematic information regarding positions of one or more joints in the respective repositionable arms 112 and/or 122 and/or the respective imaging device 114 and/or instrument 124; positions and/or orientations of imaging device 114 and/or instrument 124; and/or the like. In some examples, the exchanged information may include one or more sensor readings, mode indications, interrupts, state transitions, events, fault conditions, safety warnings, and/or the like. In some examples, the exchanged information may include one or more images captured by imaging device 114, information derived from the one or more images, calibration and/or configuration parameters specific to instrument 124 and/or imaging device 114, and/or the like.

According to some embodiments, imaging device 114 may alternatively be mounted to repositionable arm 122 separately from where an instrument 124 would be mounted using a repositionable device interface, such as on a link of repositionable arm 122 located proximal to a link to which instrument 124 is mounted. According to some embodiments, imaging device 114 may alternatively be mounted to a fixture or be a hand-held imaging device, and not mounted to a repositionable arm of a computer-assisted device. According to some embodiments, imaging device 114 may alternatively be mounted to a ceiling, floor, wall, and/or equipment physically separate from the repositionable arm 122. In some examples, when imaging device 114 is not mounted to a repositionable arm of a computer-assisted device, coordinate system b₀ may be the same coordinate system as coordinate system c.

In computer-assisted systems with multiple separate and/or modular computer-assisted devices and repositionable arms that do not share a common known base (e.g., embodiments like those shown in FIG. 1), or for some other reason the transform relating the computer-assisted devices and/or repositionable arms are not known even though there is a common base (e.g. one or more faulty sensors, one or more inadequate sensing systems, and/or the like), there is often a difficulty in establishing registration, or in maintaining sufficiently accurate registration, between the multiple computer-assisted devices and/or repositionable arms. This is especially important when one of the computer-assisted devices has an imaging device mounted thereto, and images captured by the imaging device are the basis for intuitive teleoperation of an instrument mounted to another repositionable arm of one of the other computer-assisted devices. In some examples, the registration may be established by determining a registration transform (e.g., registration transform ^{b}^{₀}T_{b}_{₁}) between a base coordinate system (e.g., coordinate system b₀) of the computer-assisted device to which the imaging device is mounted, and a base coordinate system (e.g., coordinate system b₁) of the computer-assisted device to which the teleoperated instrument is mounted. In some embodiments, this registration difficulty also exists when a hand-held imaging device is used, and the base coordinate system for the imaging device is not tied to a base of a repositionable structure (e.g. the base coordinate system for the imaging device may be tied to the imaging device itself). In some embodiments, the registration transform may be used for collision avoidance between computer-assisted devices, overlaying user interface elements on captured images, coordinating the motions of multiple computer-assisted devices (e.g., for autonomous task sequences), and/or the like.

Vision-based registration includes determining the registration transform by mapping positions and/or orientations of features of an object (e.g., an instrument mounted to a repositionable arm, the repositionable arm, a computer-assisted device, and/or the like) as observed, extracted, or derived from one or more images of the object and the positions and/or orientations of the features that are known or expected from the structural and/or kinematic models of the object. Because the registration transform is not always precise, the registration transform is often chosen as the registration transform that minimizes some cost function (e.g., an aggregate error, such as L₂ error) between the visually extracted features and the expected features when the registration transform is used to map the visually extracted features and the expected positions and/or orientations of the corresponding features to a common coordinate frame. Examples of registration techniques are described in greater detail in commonly-owned International Patent Application No. PCT/US2019/056443 (filed Oct. 16, 2019 and disclosing “Systems and Methods for Master/Tool Registration and Control for Intuitive Motion”) and U.S. Provisional Patent Application No. 62/957,537 (filed Jan. 6, 2020 and disclosing “System and Method for Inter-Arm Registration”), both of which are incorporated by reference.

Like many techniques, the quality and/or accuracy of vision-based registration depends significantly on the quality and/or accuracy of the data used to determine the registration transform. Images used for vision-based registration are not always ideal. In some cases, images used for registration may be noisy, such as due to focus issues, depth of field issues, movement, and/or the like. In some cases, one or more features, which may be useful for registration, may not be observable in every image, either because they are out of a field of view of the imaging device or because they are occluded by some other object, including other parts of the object for which the registration is desired. In some cases, one or more features, which may be useful for registration, may be misidentified by the image analysis techniques. In some cases, one or more features, which may be useful for registration, may not be easily identified or determined due to poor or pathological viewing angles of the object and/or the like.

To address these issues, it would be helpful to have one or more techniques that can evaluate the features extracted from images (either primary or secondary) to determine whether they are suitable and/or have sufficient integrity for use by a vision-based registration system. This helps reduce the likelihood that poor feature data will be provided to the vision-based registration system such that the resulting registration transform becomes less accurate and/or less reliable.

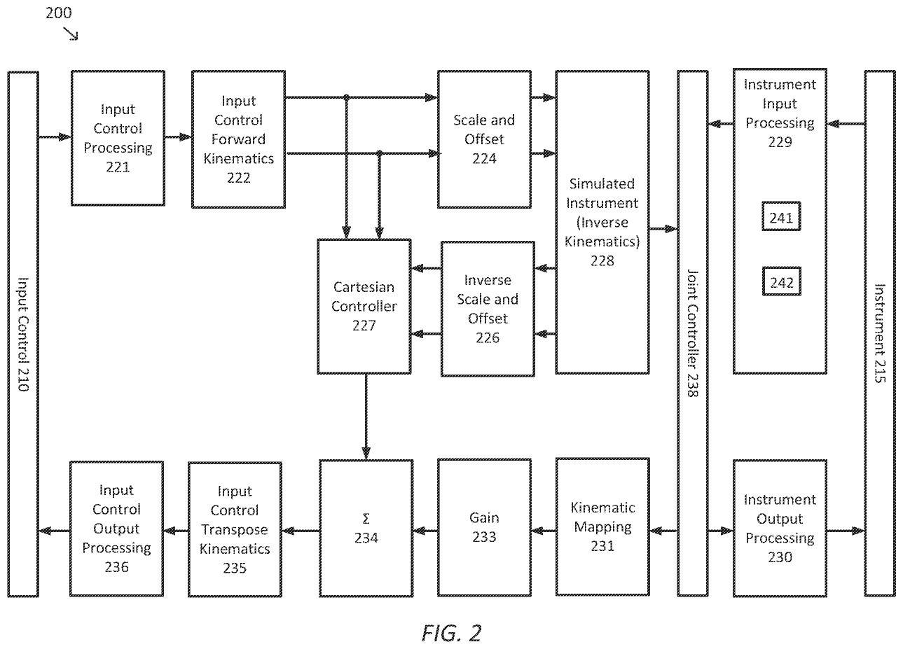

FIG. 2 is a simplified diagram of a control system 200 according to some embodiments. In some embodiments, control system 200 may be part of control module 170. In some embodiments, control system 200 may be used to control the movement of imaging device 114 and/or instrument 124 of FIG. 1. In some examples, the movement may include one or more of a position, an orientation, a pose, motion, and/or the like of imaging device 114 and/or instrument 124. In some examples, the movement may be commanded by an operator using one or more input controls. Further aspects of control system 200 are described in further detail in U.S. Patent Application Publication No. 2019/0143513, which is incorporated by reference herein.

In some embodiments, both the operator input system and the computer-assisted device used to manipulate the instrument may include a number of links connected by joints so as to facilitate control of multiple degrees-of-freedom of the instrument. As the operator moves the one or more input controls of the operator input system from a first pose to a second pose during the course of performing a procedure, sensors associated with the joints of the operator input system provide information indicating such commanded movement in the joint space of the one or more input controls. In some examples, the commanded movement includes commanded acceleration provided by sensors associated with links of the operator input system. Sensors associated with the instrument and/or the computer-assisted device provide information indicating movement of the instrument in an instrument joint space for feedback purposes.

As shown in FIG. 2, control system 200 is shown for control of an instrument 215 using an input control 210. In some examples, instrument 215 may be consistent with imaging device 114 and/or instrument 124. In some examples, control system 200 may be adapted to use multiple input controls to control instrument 215. In some examples, control system 200 may be adapted to control two or more instruments.

An input control processing unit 221 receives information of the joint positions and/or velocities of the joints in input control 210 and/or the operator input system. In some examples, the joint positions may be sampled at a control system processing rate. An input control forward kinematics processing unit 222 receives the joint positions and velocities from input control processing unit 221 and transforms them from the joint space of input control 210 to corresponding positions and velocities of a reference coordinate system associated with the operator input system. In some examples, input control forward kinematics processing unit 222 accomplishes this transformation by using a Jacobian and reference system related information. In some examples, the reference coordinate system of the operator input system may be a reference coordinate system for eyes of the operator. In some embodiments, one operating mode of control system 200 is to ensure that the motion of instrument 215 in the imaging device reference frame corresponds to the motion of input control 210 in the reference coordinate frame for the eyes of the operator.

A scale and offset processing unit 224 receives the pose, velocity, and acceleration commands from input control forward kinematics processing unit 222, scales the commanded movement according to a scale factor selected to perform a procedure, and takes into account offsets to generate a desired pose and velocity of instrument 215. The scale adjustment is useful where small movements of instrument 215 are desired relative to the larger movement of input control 210 in order to allow more precise movement of instrument 215 at a work site. The offsets determine, for example, a corresponding position and/or orientation of an instrument coordinate system (e.g., instrument coordinate system t) relative to a position and orientation of input control 210 in the reference coordinate system of the operator input system.

A simulated instrument processing unit 228 receives the desired instrument position and velocity commands from scale and offset processing unit 224 and limits the desired instrument pose, velocities, accelerations, and/or the like to assigned limits, such as to enforce correct and intuitive operation of instrument 215 by keeping instrument 215 and any associated joints within range of motion limits and/or the like. Simulated instrument processing unit 228 generates simulated instrument and computer-assisted device joint states (e.g., positions, velocities, accelerations, and/or the like). In some examples, the simulated instrument and computer-assisted device joint states are determined based on a manipulator Jacobian of instrument 215 and/or the computer-assisted device to which instrument 215 is mounted.

An inverse scale and offset processing unit 226 receives the simulated joint position and velocity commands from simulated instrument processing unit 228, and performs an inverse function (inverse to that of the scale and offset processing unit 224) on the simulated joint position and velocity commands. A Cartesian controller 227 receives the inputs to scale and offset processing unit 224 and the outputs of inverse scale and offset processing unit 226. Cartesian controller 227 then generates an error signal as a difference between the inputs to scale and offset processing unit 224 and the outputs of inverse scale and offset processing unit 226 and a Cartesian force “F_{CART}” from the error signal.

An input control transpose kinematics processing unit 235 receives the Cartesian force F_{CART} through a summation node 234, and generates a corresponding torque in joint space using, for example, the Jacobian transpose matrix and kinematic relationships associated with the operator input system. In systems where the operator input system has actuator-driven joints for range-of-motion limits or force feedback, an input control output processing unit 236 receives the output from input control transpose kinematics processing unit 235 and generates electrical signals for controlling the actuators of the operator input system and input control 210. In some examples, the control of the actuators of the operator input system and input control 210 may be felt by the operator as haptic feedback.

As input control processing unit 221 is receiving input control 210 and operator input system joint positions from sensors, an instrument input processing unit 229 is also receiving instrument positions from sensors in instrument 215 and the computer-assisted device. In some examples, the instrument positions are received by instrument input processing unit 229 at the control system processing rate. Instrument input processing unit 229 includes an actuator-side input processing unit 241 and a load-side input processing unit 242. Actuator-side input processing using 241 receives joint measurement data (e.g., pose, velocity, acceleration, and/or the like data) from actuator-side sensors in instrument 215 and/or the computer-assisted device. Load-side input processing unit 322 receives link data (e.g., position, motion, and/or the like) of the links in instrument 215 and the computer-assisted device from load-side sensors. A joint control unit 238 receives the joint measurement data and the link data from instrument input processing unit 229 and the simulated joint commands from simulated instrument processing unit 228 and generates instrument command signals for the joint actuators in instrument 215 and/or the computer-assisted device and input control feedback command signals for the joint actuators in input control 210 and/or operator input system.

The instrument command signals are generated by joint control unit 238 so as to drive joints of instrument 215 and/or the computer-assisted device until feedback errors calculated in joint control unit 238 zero out. An instrument output processing unit 230 receives the instrument command signals from joint control unit 238, converts them into appropriate electrical signals, and supplies the electrical signals to the joint actuators of instrument 215 and/or the computer-assisted device so as to drive the actuators accordingly.

The input control feedback command signals are generated by joint control unit 238 which reflect forces being exerted against instrument 215 and/or the computer-assisted device supporting instrument 215, back to the operator input system and input control 210 so that haptic feedback may be felt in some form by the operator. In some examples, joint control unit 238 may generate the input control feedback command signals based on the joint position and/or velocity tracking errors in instrument 215 and/or the computer-assisted device. A kinematic mapping unit 231 receives the input control feedback command signals from joint control unit 238 and generates the corresponding Cartesian force at the tip of instrument 215 relative to the reference coordinate system associated with the operator input system.

A gain 233 adjusts the magnitude of the Cartesian force so as to ensure system stability while providing adequate force sensation to the operator. The gain adjusted Cartesian force is then passed through summation node 234 and processed along with the Cartesian force provided by Cartesian controller 227 through input control transpose kinematics processing unit 235 and input control output processing unit 236 as previously described.

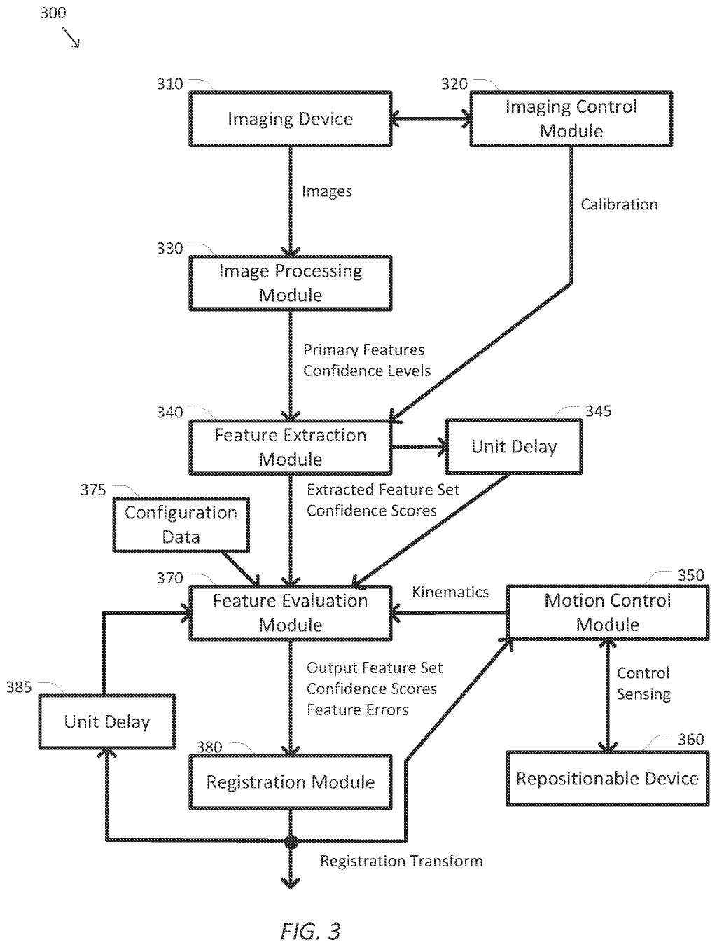

FIG. 3 is a simplified diagram of a data flow model of an image-based registration system 300 according to some embodiments. In some embodiments, all or part of image-based registration system 300 may be part of control module 170. As shown in FIG. 3, image-based registration system 300 includes an imaging device. In some examples, imaging device is consistent with imaging device 114. Imaging device 310 captures one or more images of a workspace. The images may include images of a repositionable device 360 for which registration between imaging device 310 and repositionable device 360 is desired. In some examples, repositionable device 360 is consistent with the instrument 124. In some examples, the images may be monoscopic images, stereoscopic images, monoscopic images with depth information, and/or the like. The images are passed to an image processing module 330 for further processing. Imaging device 310 is operated under the control of an imaging control module 320.

Imaging control module 320 is responsible for control of imaging device 310. In some examples, imaging control module 320 may send one or more commands to imaging device 310 to control the operation of imaging device 310. In some examples, the one or more commands may include one or more commands to control a position, an orientation, or a pose of imaging device 310. In some examples, the one or more commands may include one or more commands to control optical properties of imaging device 310, such as one or more of focus, field of view, zoom, aperture, and/or the like. In some examples, the one or more commands may include one or more commands to control when imaging device 310 captures images, how often imaging device 310 captures images, and/or the like. In some examples, imaging control module 320 may further provide intrinsic and/or extrinsic imaging device calibration parameters and/or similar information about imaging device 310 to a feature extraction module 340 to aid feature extraction module 340 during feature extraction.

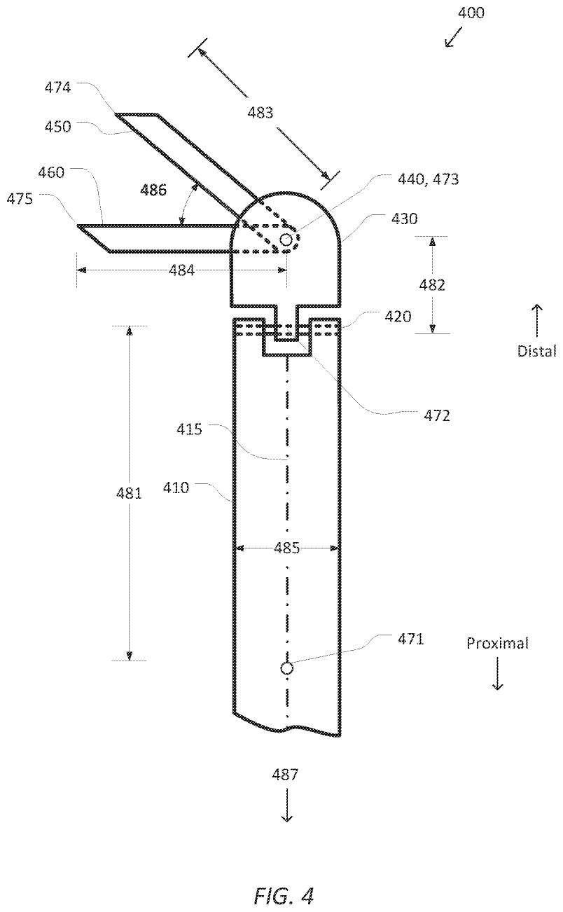

Image processing module 330 receives the images captured by imaging device 310. In some examples, image processing module 330 analyzes the captured images to identify primary features of repositionable device 360 that may be used for registration. In some examples, image processing module 330 may analyze the captured images in image space or pixel space to identify the primary features. In some examples, the primary features may correspond to points, edges, axes, fiducials, indicia, and/or the like observable in the captured images. Examples of primary features that are observable in the captured images are shown in FIG. 4, which is a simplified diagram of a distal end of an instrument 400 according to some embodiments. In some embodiments, instrument 400 may be consistent with instrument 124.

As shown in FIG. 4, instrument 400 includes an elongate shaft 410, for which the distal portion is shown as may be consistent with the portion of instrument 400 that may be captured in images obtained by imaging device 310 and/or imaging device 114. Instrument 400 further includes an articulated wrist, which includes a wrist link 430 coupled to a distal end of shaft 410 via a pivoting mechanism, such as a first clevis pin 420. First clevis pin 420 allows wrist link 430 to rotate in a degree of freedom relative to shaft 410. In some examples, wrist link 430 and first clevis pin 420 allow rotation about a yaw degree of freedom.

Instrument 400 further includes a first jaw 450 and a second jaw 460, which are independently rotatable relative to wrist link 430 via a pivoting mechanism, such as a second clevis pin 440. First jaw 450 and second jaw 460 may be used to grasp a material, such as tissue in a medical example. In some examples, first jaw 450 and second jaw 460 may be rotated together to provide a pitch degree of freedom. In some examples, first jaw 450 and/or second jaw 460 may further include one or more of a stapling mechanism, a cutting mechanism, an energy delivery mechanism, and/or the like (not shown).

One way to characterize an instrument, such as instrument 400 and/or repositionable device 360, having one or more degrees of freedom (DOFs) is through a skeleton that includes points and line segments between points. In some examples, the instrument may be characterized using a solid model that uses pre-defined shapes to approximate the volume of the instrument, a surface model, a wireframe or mesh model, and/or the like. In some examples, the skeleton includes a 3D model of instrument 400 and/or repositionable device 360. In some examples, the skeleton is developed from one or more primary features and/or from combinations of two or more features of instrument 400 and/or repositionable device 360. In some examples, the primary features may correspond to points on instrument 400 and/or repositionable device 360 that can be extracted from the image. In some examples, the combinations of two or more features may correspond to line segments, line lengths, angles, and/or the like.

FIG. 4 also shows several features of instrument 400 that correspond to 2D primary features that may be extracted from images captured of instrument 400 from one or more views (such as a left- and right-view from a stereoscopic imaging device) and/or to 3D features derived from left and right images captured of instrument 400 and/or from images captured from instrument 400 in conjunction with depth and/or ranging information. As shown, proximal shaft feature 471 corresponds to a proximal position on shaft 410. In some examples, proximal shaft feature 471 may be identified based on a fiducial, indicia, a characteristic shape, and/or the like on shaft 410 located away from the distal end of shaft 410. In some examples, shaft feature 471 may be consistent with a point at the centroid of a cross-section of shaft 410 at an arbitrary distance from first clevis pin 420. In some examples, shaft feature 471 may be consistent with a point on the edge of shaft 410 at an arbitrary distance from first clevis pin 420. A first clevis feature 472 corresponds to a location of first clevis pin 420 where first clevis pin 420 couples shaft 410 to wrist link 430. In some examples, first clevis feature 472 may be identified based on a fiducial, indicia, a characteristic shape and/or the like located near the distal end of shaft 410 and/or the proximal end of wrist link 430. In some examples, first clevis feature 472 may be consistent with a geometrical center of first clevis pin 420, such as a point lying on a centerline of the first clevis pin 420 and equidistant from either end of first clevis pin 420. A second clevis feature 473 corresponds to a location of second clevis pin 440 where second clevis pin 440 couples first jaw 450 and/or second jaw 460 to wrist link 430. In some examples, second clevis feature 473 may be identified based on a fiducial, indicia, a characteristic shape and/or the like located on wrist link 430. In some examples, second clevis feature 473 may be consistent with the geometrical center of second clevis pin 440, such as a point lying on the centerline of second clevis pin 440 and equidistant from either end of second clevis pin 440. In some examples, the edges of the shaft 410 may be extracted to then determine a centerline 415 of shaft 410 by determining a mean of the edge lines.

A first jaw tip feature 474 corresponds to a location near a distal end of first jaw 450 and a second jaw tip feature 475 corresponds to a location near a distal end of second jaw 460. In some examples, first jaw tip feature 474 and/or second jaw tip feature 475 may be identified based on a fiducial, indicia, a characteristic shape and/or the like located at or near the distal end of first jaw 450 or second jaw 460, respectively. In some examples, jaw tip feature 474 and/or jaw tip feature 475 may be consistent with the most distal point on an axis of symmetry of the gripper formed by first jaw 450 and second jaw 460.

In addition to features that correspond to visually detectable elements of instrument 400, additional features of instrument 400 may be extracted and/or derived from one or more of the primary features and/or other extracted and/or derived features. In some examples, these derived features may be more useful for registration, because they embody more than a position of a point on instrument 400. A distal shaft length feature 481 corresponds to a distance between proximal shaft feature 471 and first clevis pin 420 along a centerline of shaft 410. In some examples, when the distal portion of shaft 410 or the extent of the shaft between fiducial markers corresponding to proximal shaft feature 471 and first clevis feature 472 is fixed in length, distal shaft length feature 481 is a kinematic-invariant feature. However, in some examples, when the distal portion of shaft 410 includes a linear telescoping joint (not shown), distal shaft length feature 481 is a kinematic-variant feature as it depends on a position of the linear telescoping joint. Additional examples, of other length-based features include a wrist link length feature 482 between first clevis feature 472 and second clevis feature 473, a first jaw length feature 483 between second clevis feature 473 and first jaw tip feature 474, and a second jaw length feature 484 between second clevis feature 473 and second jaw tip feature 475. Another example of a distance-based feature includes a shaft diameter feature 485 corresponding to a diameter of shaft 410 perpendicular to a longitudinal axis of shaft 410 (e.g., the line between proximal shaft feature 471 and first clevis feature 272). The various length features 482-485 are also examples of kinematic-invariant features.

Orientation-based features are also possible. For example, an orientation direction of the longitudinal axis of shaft 410 corresponding to centerline 415, an orientation of first jaw 450, an orientation of second jaw 460, and/or the like may be used as corresponding kinematic-variant features.

Angle-based features are also possible. For example, a jaw angle feature 486 corresponds to an angle formed by first jaw tip feature 474, second clevis feature 473, and second jaw tip feature 475. Another example of an angle feature (not expressly shown) includes jaw plane angle feature, which is the angle between the line joining proximal shaft feature 471 and first clevis feature 472 and the plane formed by first jaw tip feature 474, second clevis feature 473, and second jaw tip feature 475. Jaw angle feature 486 and the jaw plane angle feature are examples of kinematic-variant features. In some examples, kinematic-invariant angle features are possible where the relationships between two portions of instrument 400 include a fixed angular relationship.

In some examples, additional features may be determined even when they may not be within the field of view of imaging device 310. In some examples, when instrument 400 is operated about a remote center of motion that allows shaft 410 to be articulated about the remote center of motion, but does not allow the remoter enter of motion to move in position, a remote center of motion feature 487 may be determined by knowing an insertion degree of freedom for instrument 400 and shaft 410 and projecting back from proximal shaft feature 471 and/or first clevis feature 472 and/or by using orientation information for shaft 410 obtained over multiple images over time to find a common intersection point along the longitudinal axis of shaft 410 from each of the images. In some examples, such projections over multiple images may not intersect at a unique intersection point, and therefore the point closest to the different projection lines at a certain pre-determined distance from first clevis feature 472 may be used as remote center of motion feature 487. In some examples, where such projections over multiple images may not intersect at a unique intersection point, the point with the minimum aggregate perpendicular distance to the projection lines may be used as remote center of motion feature 487. In some examples, when the remote center of motion may be adjusted (e.g., when it is temporarily not a position-invariant feature) (such as by one or more commands received from an operator), the projection lines used to determine remote center of motion feature 487 may be discarded with new projection lines after the remote center of motion becomes position-invariant being used to redetermine remote center of motion feature 487.

In some examples, a combination of each of the features 471-475 and/or 481-487 may define an instrument skeleton for instrument 400 as described above.

As discussed above and further emphasized here, FIG. 4 is merely an example which should not unduly limit the scope of the claims. One of ordinary skill in the art would recognize many variations, alternatives, and modifications. According to some embodiments, different features may be used for different repositionable devices. In some examples, a computer-assisted device having multiple repositionable devices may have one or more features corresponding to geometric relationships between the repositionable device, such as angle of a joint used to separate one repositionable device from another, a curvature of kinematic structure used to separate one repositionable device from another, and/or the like. Examples of computer-assisted device with multiple repositionable devices are described in further detail in U.S. Pat. Nos. 8,620,473 and 8,545,515, which are incorporated by reference herein.

Referring back to FIG. 3, image processing module 330 may use one or more image processing techniques to extract the various features from the images received from imaging device 310, such as any of the features described with respect to FIG. 4 and/or other similar types of features of repositionable device 360 and/or other instruments. In some examples, the one or more image processing techniques may include one or more of shape recognition (e.g., to identify any of features 471-475), edge detection (e.g., to identify any of features 471-475 and/or 485), machine learning modules, neural network modules, and/or the like. In some examples, a deep neural network may be trained to detect aspects of the left and right images from a stereoscopic imaging device that are consistent with the features described in FIG. 4.

In some embodiments, image processing module 330 may not be able to identify each of the features of repositionable device 360. In some examples, one or more of the features may not be observable in an image received from imaging device 310. In some examples, one or more of the features may be outside of a field of view, occluded by repositionable device 360 and/or some other object, and/or the like. For example, second jaw tip feature 475 may be occluded by first jaw 450 and/or wrist link 430, proximal shaft feature 471 may be outside of the field of view of imaging device 310, and/or the like. In some examples, when each of the features that are used to determine a length or angle feature are not accurately known, it may not be possible to accurately determine (or not determine at all) a length, orientation, or angle feature. For example, a determination of jaw angle feature 486 may not be as accurate if one or more of second clevis feature 473, first jaw tip feature 474, and/or second jaw tip feature 475 are not visible in an image, however, it may be possible to determine jaw angle feature 486 from a partial view of first jaw 450 and second jaw 460. In some embodiments, other imaging issues, such as focus, depth of field, poor viewing angles, and/or the like may also impact the accuracy of one or more features extracted from the images received from imaging device 310.

In some embodiments, the one or more image processing techniques may also provide a confidence level or measure, a likelihood, a probability measure, and/or the like identifying how confident image processing module 330 is in its identification of each of the primary features extracted from the images received from imaging device 310. In some examples, the confidence level may be a statistical confidence interval, a classification and/or prediction accuracy of a machine learning module, a model accuracy, an empirically determined non-parametric confidence interval, and/or the like. In some examples, the confidence level may be designed to correlate with the accuracy of predicting the corresponding primary feature in the image, such that when the accuracy is highest, the confidence level is at its maximum value. In some examples, the confidence may alternatively be designed so it is inversely correlated to the accuracy of prediction and this would not substantively change various embodiments described herein.

Image processing module 330 then provides the primary features extracted from the images and the corresponding confidence levels to a feature extraction module 340. In some examples where a stereoscopic imaging device is used to capture images of the repositionable device, a confidence level may be associated with the primary features detected from each of the left and right images. In some examples, the primary features that are consistent with the same feature (such as any feature described in the examples of FIG. 4) may have different confidence levels for detection in the left and the right images.

Feature extraction module 340 receives the primary features and confidence levels from image processing module 330 and uses them to generate a feature set for repositionable device 360. In some examples, these features may correspond to geometrical properties of repositionable device 360, such as link lengths, link widths, angles, and/or the like. In the examples of FIG. 4, the extracted feature set may correspond to some or all of the features 471-475 and/or 481-487.

In some examples, feature extraction module 340 may use one or more models (e.g., one or more geometric and/or kinematic models) of repositionable device 360 to determine the extracted features based on the features provided by image processing module 330. In some examples, feature extraction module 340 may use one or more modeling algorithms, feature extraction algorithms, machine learning modules, neural network modules, and/or the like to extract the features of repositionable device 360. Examples of feature extraction techniques are also described in greater detail in commonly-owned International Patent Application No. PCT/US2019/056443 (filed Oct. 16, 2019 and disclosing “Systems and Methods for Master/Tool Registration and Control for Intuitive Motion”) and U.S. Provisional Patent Application No. 62/957,537 (filed Jan. 6, 2020 and disclosing “System and Method for Inter-Arm Registration”), both of which are incorporated by reference.

In some embodiments, feature extraction module 340 may rely on information from multiple images over time to determine one or more features, such as a remote center of motion feature 487 described with respect to FIG. 4. In some examples, feature extraction module 340 may use multiple images to determine one or more position-invariant, rotation-invariant, and/or pose-invariant features. In some examples, a currently extracted position-invariant, rotation-invariant, and/or pose-invariant feature may be aggregated with previous extracted values for the same feature to obtain a better estimate of the extracted position-invariant, rotation-invariant, and/or pose-invariant feature. In some examples, the aggregation may include an average, an average over a moving window, exponential smoothing, and/or the like. In some examples, a position-invariant feature may correspond to a kinematically-constrained feature (e.g., remote center of motion feature 487), a targeting fiducial that does not move, a kinematically-constrained feature, and/or the like. In some examples, a rotation-invariant feature may correspond to a kinematically-constrained feature (e.g., a position of a cannula clamping feature that may be constrained to rotate about a cannula axis), a targeting fiducial that may be reached from more than one orientation, and/or the like. In some examples, a pose-invariant feature may correspond to a kinematically-constrained feature (e.g., a positionally and rotationally constrained cannula dock), a targeting fiducial, and/or the like. In some examples, a pose-invariant feature may correspond to a combination of a position-invariant feature and a rotation-invariant feature. In some examples based on FIG. 4, feature extraction module 340 may use information about the position and orientation of shaft 410 over multiple images to determine a location of remote center of motion feature 487. In some examples, remote center of motion feature 487 may correspond to a common intersection point from the proximal projections of the longitudinal axis of shaft 410 in each of images. In some examples, feature extraction module 340 may use a clustering or minimization algorithm to identify a point in space that, in aggregate, has a smallest perpendicular distance to each of projected longitudinal axes. This point may then correspond to the remote center of motion feature.

In some embodiments, feature extraction module 340 may generate a corresponding confidence score for each of the features in the extracted feature set. In some examples, the confidence score may be based, at least in part, on the confidence levels for each of the primary features used to determine an extracted feature and/or on the confidence scores of previously extracted features used to extract a corresponding feature. As an example from FIG. 4, when proximal shaft feature 471 and first clevis feature 472 are used to determine distal shaft length feature 481, the confidence score for distal shaft length feature 481 may be determined based on a combination of the confidence levels and/or confidence scores for proximal shaft feature 471 and first clevis feature 472. In some examples, the confidence levels and/or confidence scores for proximal shaft feature 471 and first clevis feature 472 may be combined with a function that performs a conjunctive operation, such as a triangular norm function. Examples of triangular norm functions include minimum, algebraic product, bounded difference, drastic intersection, and/or the like. In some examples, the one or more modeling algorithms, feature extraction algorithms, machine learning modules, neural network modules, and/or the like may generate the corresponding confidence scores. In some examples, the feature set may further include 3D feature versions of the primary features provided by image processing module 330, such as by triangulating between left and right images of a stereoscopic imaging device to determine the 3D feature from the 2D primary features in the left and right images and/or using depth or ranging information. Feature extraction module 340 then provides the extracted feature set and the corresponding confidence scores to a feature evaluation module 370. In some examples, feature extraction module 340 may also provide the extracted feature set and the corresponding confidences scores from a previous iteration through image-based registration system 300 (e.g., as indicated by a unit delay 345) to support a differential shape analysis as is described in further detail below. In some examples, the confidence level of predicting a 2D primary feature in the imaging plane may be available as a spatial or other probability distribution which may be used to compute a confidence score of a 3D secondary feature as an a posteriori probability distribution. Examples of the use of probability distributions to determine the confidence score of a 3D secondary feature from 2D primary features are described in further detail in Hartley, et al., “Multiple View Geometry in Computer Vision,” Second Ed., 2003, which is incorporated by reference herein.

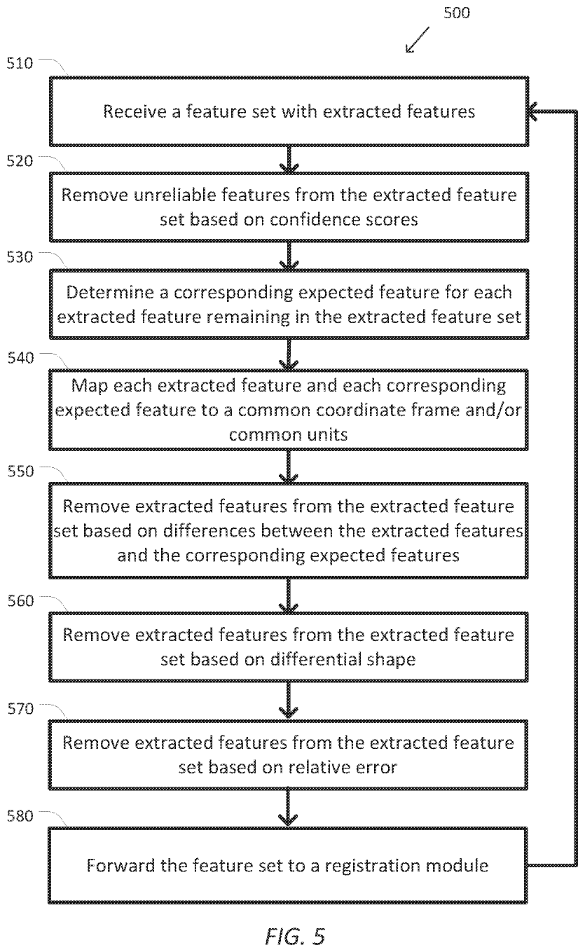

Feature evaluation module 370 receives the extracted feature set and corresponding confidence scores to determine which, if any, of the extracted features in the extracted feature set are to be provided to a registration module. In some examples, feature evaluation module 370 may further use kinematic information about repositionable device 360 received from a motion control module 350 to aid in the evaluation of each of the features in the extracted feature set. In some examples, the kinematic information may be utilized to determine the expected features for one or more of the pose-variant features described in the examples of FIG. 4. In some examples, the kinematic information may be time synchronized to the images used to determine the extracted feature set. In some examples, feature evaluation module 370 may further use a registration transform from a previous iteration through image-based registration system 300 received from registration module 380 through a unit delay 385. In some examples, feature evaluation module 370 may use a part properties file and/or other configuration data 375 to determine expected features for one or more of the kinematic-invariant features of repositionable device 360. As part of its evaluation of the extracted feature set, feature evaluation module 370 may determine whether to remove one or more features from the extracted feature set and then remove those one or more features from the extracted feature set to generate an output feature set and/or adjust the confidence score for one or more of the features in the output feature set to control how much reliance registration module 380 places on each of the features in the output feature set. Removal of a feature from the output feature set can comprise actual removal, such that the feature is not included in the output feature set. Removal of a feature from the output feature set can also comprise constructive removal, such as by setting a variable or flag in a manner that causes the contribution of the feature on the result (e.g., the registration) to be so small that the result with constructive removal is identical or substantially identical, and has the same effect as the result with actual removal. For example, feature evaluation module 370 could set a confidence score for a feature to a zero or near zero value (e.g., a value below a nominal confidence score, such as 0.05, 0.1, 0.5, and/or the like where a nominal confidence score is 1.0) to indicate to registration module 380 that that feature should be given little weight when determining registration and, thus, constructively remove that feature from the output feature set. In some examples, feature evaluation module 370 may further pass on the corresponding confidence score information on each of the features to registration module 380. In some examples, feature evaluation module 370 may also generate corresponding error information for each of the features in the output feature set that describes a feature error or feature difference between an extracted feature in the output feature set and a corresponding expected feature for the extracted feature. Feature evaluation module 370 then passes the output feature set, the corresponding confidence scores, and the corresponding feature errors to registration module 380. The operation of feature evaluation module 370 is described in further detail in FIG. 5.

Motion control module 350 is responsible for operating repositionable device 360, such as under the supervision of one or more operators. In some examples, motion control module 350 may receive one or more commands from an operator in an imaging device coordinate frame, map the commands to a coordinate frame of repositionable device 360 using the registration transform from registration module 380. Motion control module 350 uses the mapped commands to control one or more joints of repositionable device 360. Motion control module 350 also senses a state of repositionable device 360 (e.g., one or more joint positions) to aid in the control of repositionable device 360 and/or to supply kinematic information to feature evaluation module 370. In some embodiments, motion control module 350 may be consistent with and/or include portions of control system 200.

Repositionable device 360, which in some instances comprises part or all of a repositionable structure, corresponds to the device to be registered with imaging device 310. Repositionable device 360 is controlled by motion control module 350, such as based on commands received from one or more operators. In some embodiments, repositionable device 360 may be consistent with and/or include part or all of computer-assisted device 120, repositionable arm 122, instrument 124, and/or instrument 400.

Registration module 380 receives the output feature set, the corresponding confidence scores, and/or the corresponding feature errors and generates and/or updates the registration transform between imaging device 310 and repositionable device 360. In some examples, registration module 380 may use the corresponding feature errors to determine an error in a previously determined registration transform, such as the registration transform provided to feature evaluation module 370. The error in the previously determined registration transform may then be used to determine an update to improve the registration transform. In some examples, the update may apply to a translation portion and/or a rotation portion of the registration transform. Examples of registration techniques usable by registration module 380 are described in greater detail in commonly-owned International Patent Application No. PCT/US2019/056443 (filed Oct. 16, 2019 and disclosing “Systems and Methods for Master/Tool Registration and Control for Intuitive Motion”) and U.S. Provisional Patent Application No. 62/957,537 (filed Jan. 6, 2020 and disclosing “System and Method for Inter-Arm Registration”), both of which are incorporated by reference.

As discussed above and further emphasized here, FIG. 3 is merely an example which should not unduly limit the scope of the claims. One of ordinary skill in the art would recognize many variations, alternatives, and modifications. According to some embodiments, different arrangements of the various modules and/or the information passed between the modules are possible. In some examples, image processing module 330 and feature extraction module 340 may be a combined module that receives the images from imaging device 310 and generated the extracted feature set and corresponding confidence scores. In some examples, image processing module 330 and/or feature extraction module 340 may include multiple sub-modules that are used to extract one or more specific features to be included in the feature set. In some examples, any of the corresponding confidence levels, corresponding confidence scores, and/or corresponding feature errors may not be shared with other modules.

In some embodiments, the image-based registration system 300 may operate in a loop so that as additional images are captured by imaging device 310 and analyzed using image processing module 330, feature extraction module 340, feature evaluation module 370, and/or registration module 380 the registration transform may be periodically updated. In some examples different modules in image-based registration system 300 may be running at different sample rates. In some examples, the kinematics data passed from motion control module 350 to feature evaluation module 370 may be sampled at a rate much faster than the sampling rates of blocks 310-340. In some examples, such disparity in sampling rate may be managed by sub-sampling, filtering, or other techniques such that the most recent time-synchronous and smoothed data is available to feature evaluation module 370. In some examples, unit delay 345 and/or unit delay 385 may be determined based on one or more of the sampling rates.

According to some embodiments, image-based registration system 300 may include two or more repositionable devices. In some examples, when two or more of the repositionable devices share a common coordinate frame (e.g., a device with multiple repositionable arms and/or instruments), a common feature set including features for each of the repositionable devices may be used to support registration to those repositionable devices. In some examples, separate feature sets and registration transforms may be used for repositionable devices that do not share a common coordinate frame with image processing module 330, feature extraction module 340, feature evaluation module 370, and/or registration module 380 processing the features and/or registration transform separately for each of the repositionable devices.

FIG. 5 is a simplified diagram of a method 500 of feature integrity checking according to some embodiments. One or more of the processes 510-580 of method 500 may be implemented, at least in part, in the form of executable code stored on non-transitory, tangible, machine readable media that when run by one or more processors (e.g., the processor 150 in control unit 140) may cause the one or more processors to perform one or more of the processes 510-580. In some embodiments, method 500 may be performed by one or more modules, such as control module 170 and/or feature evaluation module 370. In some embodiments, method 500 may be used to evaluate a set of features extracted from images to determine which, if any, of the extracted features are suitable for use by a registration module that determines a registration between an imaging device used to obtain the images and a repositionable structure visible in the images. In some embodiments, one or more of processes 520, 560, and/or 570 are optional and may be omitted. In some embodiments, method 500 may include additional processes, which are not shown.

At a process 510, a feature set with extracted features is received. In some examples, the feature set includes one or more features of a repositionable structure, such as part or all of repositionable device 360, instrument 400, instrument 124, repositionable arm 122, and/or computer-assisted device 120 that have been extracted from one or more images of the repositionable structure. In some examples, the feature set may define part or all of a skeleton of the repositionable structure. In some examples, the extracted feature set may be modeled as a feature vector with an entry for each feature, a set of (key, value) pairs, and/or the like. In some examples, each key in a (key, value) pair may uniquely identify the corresponding extracted feature in the value of the (key, value) pair. In some examples, the key may be a unique integer, a unique character string, and/or the like.

In some examples, each of the one or more extracted features may correspond to a 3D version of a primary feature, a length feature, an angle feature, an orientation feature, and/or the like. In some examples, each of the extracted features may correspond to a kinematic-invariant and/or a kinematic-variant feature of the repositionable structure. In some examples, one or more of the extracted features may correspond to a position-invariant, rotation-invariant, or pose-invariant feature. In some examples, one or more of the features may correspond to one of the features described in the examples of FIG. 4. In some examples, each of the one or more expected features may have a corresponding confidence score identifying how likely the corresponding expected feature is to be accurate. In some examples, the feature set and corresponding confidence scores may be received or obtained from a feature extraction module, such as feature extraction module 340, and/or an image processing module, such as image processing module 330.

At an optional process 520, any unreliable features in the extracted feature set are removed based on the corresponding confidence scores. In some examples, any of the extracted features in the feature set whose corresponding confidence score is below a corresponding confidence score threshold is removed from the extracted feature set and is no longer considered. In some examples, the corresponding confidence score threshold may be based on an aggregate confidence score for the feature set as a whole. In some examples, the corresponding confidence score threshold may be different for different extracted features.

At a process 530, a corresponding expected feature is determined for each extracted feature remaining in the extracted feature set. In some examples, the corresponding expected feature for an extracted feature may be determined from one or more models of the repositionable structure and/or kinematic information of the repositionable structure. In some examples, the kinematic information may be received from a motion control module, such as motion control module 350 and/or from configuration data, such as configuration data 375. Referring to the examples of FIG. 4, distal shaft length feature 481 is a kinematic-invariant feature for which an expected feature (e.g., the length) can be known from the one or more geometric models of instrument 400. Similarly, expected features for wrist link length feature 482, first jaw length feature 483, second jaw length feature 484, and/or shaft diameter feature 485 have corresponding expected features that can be obtained from the one or more geometric models of instrument 400. In contrast, jaw angle feature 486 is a kinematic-variant feature whose expected value can be determined from the one or more geometric models of instrument 400 and kinematic information (e.g., joint positions) of the two joints used to rotate first jaw 450 and second jaw 460 relative to wrist link 430 about second clevis pin 440. Similarly, an expected feature for the jaw plane angle feature can be determined from the one or more geometric models of instrument 400 and kinematic information (e.g., joint position) of the joint used to rotate wrist link 430 relative to shaft 410 about first clevis pin 420. Similarly, kinematic information may be used to determine corresponding expected features for orientation expected features. In some examples, a similar approach may be used for position-invariant, rotation-invariant, and/or pose-invariant extracted features (e.g., remote center of motion feature 487), which may be determined from the one or more geometric and/or kinematic models of instrument 400, one or more geometric and/or kinematic models of the repositionable arm to which instrument 400 is mounted, and kinematic information. In some examples, the expected feature may be characterized using a solid model that uses pre-defined shapes to approximate the volume of the repositionable structure, a surface model, and/or a wireframe or mesh model of the repositionable structure.

At a process 540, each extracted feature and each corresponding expected feature is mapped to a common coordinate frame and/or to common units. The common coordinate frame and/or common units allows for a more direct comparison between each extracted feature and its corresponding expected feature. In some examples, an extracted feature obtained from one or more images may not be in the same units as a corresponding expected feature. In some examples, an extracted feature obtained from one or more 2-D images may appear different (e.g., a length may appear shorter in the images) as viewing angles of the imaging device changes. In some examples, an extracted feature and a corresponding expected feature that correspond to a scalar feature (e.g. length and/or an angle) may be compared as long as they have common units without having to be transformed to a common coordinate system. In some examples, such a feature may be converted to a common unit based on a ratio of the scaling factors between the coordinate frame in which images are captured by the imaging device and a coordinate frame of the repositionable structure. In some examples, the common coordinate frame may be a coordinate frame associated with the imaging device and/or the computer-assisted device to which the imaging device may be mounted, a coordinate frame associated with the repositionable structure and/or the computer-assisted device to which the repositionable structure is mounted, and/or a separate coordinate frame. In some examples, the separate coordinate frame may correspond to a world coordinate frame, a workspace coordinate frame, an inertial coordinate frame, a tracking system coordinate frame, and/or the like. In some examples, a current registration transform, may be used to map each extracted feature and/or each corresponding expected feature to the common coordinate frame. In some examples, where the extracted feature corresponds to a 3D version of a primary feature in an imaging plane of an imaging coordinate system of the imaging device (e.g., imaging device 310), the expected feature may be projected to a plane consistent with an imaging plane of the imaging coordinate system of the imaging device, by using a current registration transform, calibration parameters of the imaging device and/or the like. In some examples, the current registration transform is consistent with the registration transform provided by registration module 380. In some examples, the mapping may change one or more of a position, an orientation, and/or a size of an extracted feature or a corresponding expected feature. In some example, the change in size may be due to a difference in scaling factor between the coordinate frame in which images are captured by the imaging device and the coordinate frame of the repositionable structure. In some examples, the change in size may convert the extracted feature and the corresponding expected feature to a common unit.

At a process 550, extracted features are removed from the extracted feature set based on differences between the extracted features and the corresponding expected features. The differences are determined for each of the extracted features remaining in the feature set and the corresponding expected feature in the common coordinate frame. Referring to the examples of FIG. 4, a difference between the extracted distal shaft length feature 481 and its corresponding expected feature (e.g., from the one or more geometric models of instrument 400) may be determined by subtracting the extracted distal shaft length feature 481 and its corresponding expected distal shaft length and, optionally, taking an absolute value and/or determining a percentage difference. If it is determined that the difference in length is above a length threshold and/or the percentage difference is above a percentage difference threshold, then distal shaft length feature 481 is actually removed from the extracted feature set and/or is constructively removed from the extracted feature set (such as by setting the confidence score for distal shaft length feature 481 is set to a zero or near zero value). A similar test may be performed for wrist link length feature 482, first jaw length feature 483, second jaw length feature 484, and/or shaft diameter feature 485. Additionally, similar tests may be performed for jaw angle feature 486 and/or the jaw plane angle feature. For extracted features that include a vector of values, such as an orientation feature, remote center of motion feature 487, a position-invariant feature, a rotation-invariant feature, and/or a pose-invariant feature, a distance or magnitude metric such as L₂ and/or the Euclidean distance may be used to determine a difference, which may then be compared to a difference threshold to determine whether the feature should be removed (e.g., actually or constructively removed) from the extracted feature set. In some examples, the difference threshold may be different for different extracted features. In some examples, the difference between the extracted feature and the expected feature may be associated with the extracted feature as a feature error for the extracted feature. In some examples where the extracted and expected features are represented as solid models, the difference between them may be determined as a volumetric error. In some examples where the extracted and expected features are represented as surface models, the difference between them may be determined as error in a surface tangent, a surface normal, and/or the like.

At an optional process 560, extracted features may be removed from the extracted feature set based on differential shape. The differential shape test uses information about possible changes in the repositionable structure between two images of the repositionable structure. For example, a kinematic-invariant, a position-invariant, a rotation-invariant, and/or pose-invariant extracted feature is not expected to change between two images. In contrast, a kinematic-variant extracted feature may change based on changes in one or more joint positions, which may be determined from the kinematic information associated with each of the images used to generate the respective extracted feature sets. Thus, an expected change in an extracted feature between two images (e.g., a difference between the expected feature for a first of the two images and the expected feature for a second of the two images) may be compared to an actual change between the extracted feature in the two images (e.g., a difference between the extracted image in the first of the two images and the extracted feature in the second of the two images) to determine whether the extracted feature has changed too little or too much between the two images to be reliable and should be removed (e.g., actually or constructively removed) from the extracted feature set. In some examples, the time interval used for the differential shape for the extracted features and the time interval used for the corresponding expected features are comparable or approximately equal. In some examples, when the time intervals are not comparable, an appropriate scaling factor may be applied while comparing the differential shape for the extracted and the differential shape for the corresponding expected features. When it is determined that the differential shape indicates that a difference between the change in the extracted feature and the expected change in the extracted feature is above a difference threshold, then the extracted feature is removed from the extracted feature set. In some examples, the difference between the change in the extracted feature and the expected change in the extracted feature may alternatively be scaled based on the expected change and compared to a percentage difference threshold. In some examples, the difference threshold may be different for different extracted features. In some examples, the difference between the change in the extracted feature and the expected change in the extracted feature may be associated with the extracted feature as a feature error for the extracted feature.

In some embodiments, differential shape tests may also be applied to position-invariant, rotation-invariant, and/or pose-invariant extracted features. For example, and consistent with the description above, when it is determined that the extracted remote center of motion feature 487 changes above its corresponding difference threshold between the two images, the extracted remote center of motion feature 487 is removed (e.g., actually or constructively removed) from the extracted feature set. In some examples, this approach may also be used to determine whether the extracted remote center of motion feature 487 is to be included in the aggregation for the extracted remote center of motion feature 487 and/or included in the computations to determine the aggregate with the smallest perpendicular distance (e.g., is it an outlier or not).

At an optional process 570, extracted features are removed from the extracted feature set based on relative error. In some examples, it may be helpful to remove (e.g., actually or constructively remove) extracted features from the extracted feature set when it is determined that a feature error for those features is disproportionately higher than the feature error of other extracted features in the feature set. In some examples, the feature error for an extracted feature is compared to an aggregate feature error for the whole extracted feature set. When it is determined that the feature error for the extracted feature is above the aggregate feature error multiplied by an error factor (e.g., 1.2, 1.5, 2.0, and/or the like), the extracted feature is removed from the extracted feature set. In some examples, the aggregate feature error may be an average and/or weighted sum of the feature errors for each of the extracted features in the extracted feature set.

At a process 580, the feature set is forwarded to a registration module if any extracted features remain in the extracted feature set. In some examples, the registration module may be registration module 380 of FIG. 3. In some examples, the corresponding confidence scores and/or the corresponding feature errors for the extracted features in the extracted feature set may also be forwarded to the registration module. In some examples, the aggregate confidence score and/or the aggregate feature error may also be forwarded to the registration module. In some examples, the registration module may use the corresponding feature errors to determine an error in a previously determined registration transform, such as the registration transform used during process 540. The error in the previously determined registration transform may then be used to determine an update to improve the registration transform. In some examples, the update may apply to a translation portion and/or a rotation portion of the registration transform.

Upon completion of process 580, method 500 returns to process 510 to process a new feature set extracted from one or more new images of the repositionable structure.

In some embodiments, the corresponding confidence score thresholds of process 520; the difference thresholds of process 550, the difference thresholds of process 560, and/or the error factor of process 570 may be determined based on one or more factors. In some examples, the one or more factors include a type of the repositionable structure, a type of a repositionable arm to which the repositionable structure is mounted, a type of the imaging device used to capture the images, a task being performed with the repositionable structure, operator preference, a speed of the imaging device and/or the repositionable structure, a pose of the imaging device and/or the repositionable structure, an estimate of a pose error of the imaging device and/or the repositionable structure, a type of the extracted feature, and/or the like. In some examples, the corresponding confidence score thresholds of process 520; the difference thresholds of process 550, the difference thresholds of process 560, and/or the error factor of process 570 may be determined from the one or more factors using one or more lookup tables, one or more databases, one or more functions, and/or the like. In some examples, the type of the repositionable structure may include one or more of forceps, clip applier, a gripper, a retractor, a cautery instrument, a suction instrument, a suturing device, a stapling device, a cutting device, an energy delivery instrument and/or the like and/or a particular model of the repositionable structure. In some examples, the task being performed may correspond to clip applying, gripping, grasping, retracting, cauterizing, suction, suturing, stapling, cutting, energy application, raking a bowel, and/or the like and/or any combination of two or more such tasks. In some examples, the speed of the imaging device and/or the repositionable structure may be based on a speed of a point of interest on the repositionable structure (e.g., one or more of the primary features of the repositionable structure), a speed of a focal point of the imaging device, and/or the like. In some examples, the speed may correspond to an aggregate speed or magnitude of an aggregate velocity of multiple points of interest. In some examples, the speed may correspond to an aggregate speed or a magnitude of an aggregate velocity of one more joints in a repositionable structure of the imaging device, the repositionable structure, one or more repositionable arms, and/or the like. In some examples, a level of zooming of the imaging device may be used to determine the confidence score threshold. In some examples, the distance of the feature from the imaging device (corresponding to the working distance of the instrument in a depth direction of an imaging coordinate frame) may be used to determine the confidence score threshold. In some examples, a motion scaling between operator motions and instrument motions may be used to determine the confidence score threshold. In some examples, an importance and/or delicacy of a material near the repositionable structure in a workspace (such as vasculature in a medical example) may be used to determine the confidence score threshold. In some examples, the type of the extracted feature may be one or more of point, line, angle, primary, secondary, kinematic-invariant, kinematic-variant, position-invariant, rotation-invariant, pose-invariant, and/or the like. In some examples, primary extracted features may have relatively higher thresholds and/or error factors than secondary extracted features because primary extracted features may have to be determined with relatively higher confidence than secondary extracted features in order to support the determination of the secondary extracted features from the primary extracted features. In some examples, a first set of secondary extracted features may have relatively higher thresholds and/or error factors than a second set of secondary extracted features, which are dependent on the first set, because the first set of extracted features may have to be determined with relatively higher confidence than the second set of secondary extracted features in order to support determination of the second set of extracted features from the first set of extracted features.

As discussed above and further emphasized here, FIG. 5 is merely an example which should not unduly limit the scope of the claims. One of ordinary skill in the art would recognize many variations, alternatives, and modifications. According to some embodiments, rather than operating as filters that remove (e.g., actually or constructively remove) extracted features from the extracted feature set that fail certain tests, each of processes 520, 550, and/or 560 may operate as all-or-nothing type tests that may be used to reject the entire extracted feature set based on an aggregate test. In some examples, an aggregate score for the entire feature set may be compared against a corresponding aggregate threshold to determine whether the entire feature set, in aggregate, is too unreliable to be used for registration. In some examples, the aggregation may include an average and/or a weighted sum of the relevant factor (e.g., confidence score, difference, error, and/or the like), which is compared against a corresponding pass-fail threshold. In some examples, the weight in the aggregation for each of the extracted features may be determined based on the type of the feature and/or any of the other factors described above that may be relevant to a threshold. As an example, process 510 may determine an aggregation of the confidence scores and if the aggregation is below an aggregate confidence score threshold, then the entire feature set may be rejected with method 500 returning to process 510 to wait for a new extracted feature set to be received. Processes 550 and/or 560 may be similarly altered to determine and test aggregations of the differences and differential shape errors, respectively.

In some embodiments, when process 550 operates as an all-or-nothing type test, the extracted feature set may be partitioned into different subsets of extracted features with all-or-nothing tests being performed on each subset of extracted features. In some examples, the extracted feature set may be partitioned based on the type of each of features so that, for example, kinematic-invariant extracted features are in a different subset than kinematic-variant extracted features, which are in a different subset than position-invariant, rotation-invariant, and post-invariant extracted features. In some examples, the partitioning of the extracted feature set may be based on a perceived computational cost of performing the extracted vs. expected feature difference tests. In some examples, the computational costs for comparing extracted vs. expected features is typically lower for kinematic-invariant features than kinematic-variant features, and the computational costs for comparing extracted vs. expected features is typically lower for kinematic-variant features than position-invariant, rotation-invariant, and/or pose-invariant features. In some examples, the partitioning of the extracted feature set may be based on any technically feasible classification approach. In some examples, the partitioning may be more computationally efficient as it may be desirable to reject the entire extracted feature set if, for example, the aggregate differences for the kinematic-invariant features is too high.

According to some embodiments, method 500 may be adapted to situations where there are different configurations of computer-assisted devices, repositionable arms, and/or repositionable structure. In some examples, when additional computer-assisted devices having different base coordinate systems are present, method 500 may be applied separately for each of the additional computer-assisted devices to evaluate the extracted feature set for each of these devices. In some examples, when multiple repositionable structures share a same base coordinate system, the extracted feature set may include extracted features for each of the repositionable structures.

Some examples of control units, such as control unit 140 may include non-transitory, tangible, machine readable media that include executable code that when run by one or more processors (e.g., processor 150) may cause the one or more processors to perform the processes of method 500 and/or implement the modules of image-based registration system 300. Some common forms of machine readable media that may include the processes of method 500 and/or implement the modules of image-based registration system 300 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

1. A computer-assisted system comprising:
a repositionable arm configured to support a repositionable device; and
a control unit coupled to the repositionable arm;
wherein the control unit is configured to:
receive, from an image processing system or a feature extraction system, a feature set comprising one or more extracted features, the one or more extracted features being extracted from one or more images of a repositionable structure obtained from an imaging device, wherein the repositionable structure comprises at least one component selected from the group consisting of: the repositionable arm and the repositionable device;
determine, based on one or more models of the repositionable structure, a first expected feature, the first expected feature corresponding to a first extracted feature in the feature set;
determine an error between the first extracted feature and the first expected feature;
determine whether to remove the first extracted feature from the feature set based on the determined error;
in response to determining that the first extracted feature should be removed from the feature set, remove the first extracted feature from the feature set;
provide the feature set to a registration module; and
cause motion of the repositionable device by commanding movement of the repositionable arm based at least on a registration provided by the registration module.

2. (canceled)

3. The computer-assisted system of claim 1, wherein the repositionable structure comprises the repositionable device, wherein the computer-assisted system is a medical system, the repositionable device is a medical instrument, and wherein the imaging device is an endoscope.

4. The computer-assisted system of claim 1, wherein the repositionable structure comprises the repositionable arm or the repositionable device, the computer-assisted system further comprising:
a second repositionable arm configured to support a second repositionable device, wherein the second repositionable device comprises the imaging device.

5-7. (canceled)

8. The computer-assisted system of claim 1, wherein the first extracted feature is a kinematic-invariant feature that does not change in response to motion of the repositionable structure.

9. The computer-assisted system of claim 8, wherein the first extracted feature corresponds to:
a distance between positions of two features of the repositionable structure; or
a diameter of a part of the repositionable structure.

10. The computer-assisted system of claim 1, wherein:
the first extracted feature is a kinematic-variant feature that may be changed by motion of the repositionable structure; and
the control unit is further configured to determine the first expected feature further based on one or more joint positions of one or more joints of the repositionable structure.

11. The computer-assisted system of claim 10, wherein the first extracted feature corresponds to:
a position of a feature on the repositionable structure;
a shape of a feature on the repositionable structure;
an orientation of a part of the repositionable structure;
an angle formed by positions of three features of the repositionable structure; or
an angle formed between a line formed by positions of two features of the repositionable structure and a plane formed by positions of three or more features of the repositionable structure.

12. The computer-assisted system of claim 1, wherein the first extracted feature is a position-invariant feature, a rotation-invariant feature, or a pose-invariant feature, wherein the position-invariant feature does not change with a position of the repositionable structure, wherein the rotation-invariant feature does not change with an orientation of the repositionable structure, and wherein the pose-invariant feature does not change with the position and the orientation of the repositionable structure.

13. The computer-assisted system of claim 1, wherein the first extracted feature corresponds to a remote center of motion of the repositionable structure.

14. The computer-assisted system of claim 1, wherein to determine whether to remove the first extracted feature from the feature set based on the determined error, the control unit is configured to:
determine whether the determined error is greater than an error threshold.

15. (canceled)

16. The computer-assisted system of claim 14, wherein the control unit is further configured to determine the error threshold based on at least one type selected from a group consisting of:
a type of the repositionable structure;
a type of the repositionable arm;
a type of the imaging device;
a type of task being performed with the repositionable structure; and
a type of the first extracted feature.

17. (canceled)

18. The computer-assisted system of claim 14, wherein the control unit is further configured to determine the error threshold based on one or more parameters selected from the group consisting of:
a speed of the imaging device;
a speed of the repositionable structure;
a speed of a feature in the feature set;
a pose of the imaging device;
a pose of the repositionable structure;
an estimate of a pose error of the imaging device; and
an estimate of a pose error of the repositionable structure.

19-22. (canceled)

23. The computer-assisted system of claim 1, wherein to determine whether to remove the first extracted feature from the feature set, the control unit is configured to:
determine whether a difference is above a difference threshold, the difference being between an expected change in the first extracted feature and a change in the first expected feature, the expected change being between the feature set and a second feature set, wherein the second feature set is received from the image processing system or the feature extraction system, and wherein the second feature set comprises one or more extracted features extracted from a second image of the repositionable structure obtained from the imaging device; or
determine whether a confidence score corresponding to the first extracted feature is below a confidence score threshold.

24. (canceled)

25. The computer-assisted system of claim 1, wherein to determine whether to remove the first extracted feature from the feature set based on the determined error, the control unit is configured to:
determine whether the determined error is above an error factor times an aggregate feature error for the feature set.

26-27. (canceled)

28. The computer-assisted system of claim 1, wherein the feature set comprises a plurality of extracted features, and wherein the control unit is further configured to:
reject the feature set in response to an aggregation of a determined error for each feature in the feature set being above an aggregate error threshold.

29-31. (canceled)

32. The computer-assisted system of claim 1, wherein the control unit is further configured to:
reject the feature set in response to an aggregation of a confidence score for each feature in the feature set being below an aggregate confidence threshold, wherein the aggregation is based on a weighted sum of the confidence score of each feature, and wherein a corresponding weight used for the confidence score of each feature is based on a type of that feature.

33. The computer-assisted system of claim 1, further comprising:
the registration module;
wherein the registration module is configured to determine a registration between the imaging device and the repositionable structure based on the feature set.

34. (canceled)

35. A method comprising:
receiving, by a control unit and from an image processing system or a feature extraction system, a feature set comprising one or more extracted features, the one or more extracted features being extracted from one or more images of a repositionable structure, the one or more images being obtained from an imaging device, wherein the repositionable structure comprises at least one component selected from the group consisting of: a repositionable arm and a repositionable device supported by the repositionable arm;
determining, by the control unit based on one or more models of the repositionable structure, a first expected feature corresponding to a first extracted feature in the feature set;
determining, by the control unit, an error between the first extracted feature and the first expected feature;
determining, by the control unit, whether to remove the first extracted feature from the feature set based on the determined error;
in response to determining that the first extracted feature should be removed from the feature set, removing, by the control unit, the first extracted feature from the feature set; and
providing, by the control unit, the feature set to a registration module.

36. (canceled)

37. The method of claim 35, wherein the first extracted feature is a kinematic-invariant feature that does not change in response to motion of the repositionable structure.

38. (canceled)

39. The method of claim 35, wherein:
the first extracted feature is a kinematic-variant feature that may be changed by motion of the repositionable structure; and
the method further comprises determining, by the control unit, the first expected feature further based on one or more joint positions of one or more joints of the repositionable structure.

40-42. (canceled)

43. The method of claim 35, wherein determining whether to remove the first extracted feature from the feature set based on the determined error comprises:
determining whether the determined error is greater than an error threshold, wherein the error threshold based on a type of the first extracted feature or a task being performed with the repositionable structure.

44-52. (canceled)

53. A non-transitory machine-readable medium comprising a plurality of machine-readable instructions which when executed by one or more processors are adapted to cause the one or more processors to perform a method comprising:
receiving a feature set comprising one or more extracted features, the one or more extracted features being extracted from one or more images of a repositionable structure, the one or more images being obtained from an imaging device, wherein the repositionable structure comprises at least one component selected from the group consisting of: a repositionable arm and a repositionable device supported by the repositionable arm;
determining, based on one or more models of the repositionable structure, a first expected feature corresponding to a first extracted feature in the feature set;
determining an error between the first extracted feature and the first expected feature;
determining whether to remove the first extracted feature from the feature set based on the determined error;
in response to determining that the first extracted feature should be removed from the feature set, removing the first extracted feature from the feature set; and
providing the feature set to a registration module.
